(19) United States
(12) Patent Application Publication
(10) Pub. No.: US 2005/0192591 A1
(43) Pub. Date: Sep. 1, 2005

(54) Device for removing an elongated structure implanted in biological tissue

(76) Inventors: Chun Kee Lui, Monroeville, PA (US); Louis B. Goode, Cranberry Township, PA (US); Barry E. Norlander, Freeport, PA (US)

Correspondence Address: BRINKS HOFER GILSON & LIONE/INDY/COOK, ONE INDIANA SQUARE, SUITE 1600, INDIANAPOLIS, IN, 46204-2033, US

(21) Appl. No.: 11/067,232

(22) Filed: Feb. 25, 2005

Related U.S. Application Data
(60) Provisional application No. 60/548,754, filed on Feb. 27, 2004.

(51) Int. Cl. A61F 11/00

(52) U.S. Cl. 606/108

(57) ABSTRACT

A device for removing from a patient a previously implanted elongated structure, such as a pacemaker lead or a defibrillator lead, that may be encapsulated in biological tissue of the patient. In one form, the device comprises a sheath member having an inside dimension greater than the outside dimension of the elongated structure, and a gripping member positioned about the sheath member. The gripping member has a longitudinal passage extending substantially therethrough, which longitudinal passage is dimensioned to encircle at least a portion of the elongated structure. A proximal portion of the gripping member may extend outwardly beyond the proximal end of the elongated structure and outwardly of at least the vascular system of the patient. The gripping member may comprise a first configuration having a first diameter that is greater than the outside dimension of the elongated structure, and a second configuration having a second diameter that is substantially the same as the outside dimension of the elongated structure.

### RELATED APPLICATION

The present patent document claims the benefit of the filing date under 35 U.S.C. §119(e) of Provisional U.S. Patent Application Ser. No. 60/548,754, filed Feb. 27, 2004, which is hereby incorporated by reference.

### BACKGROUND

1. Technical Field

This invention relates generally to methods and surgical devices, and more particularly to a method and a device for separating encapsulating biological tissue from an implanted elongated structure (for example, an implanted cardiac electrical lead such as a pacemaker or defibrillator lead), and/or for removing such an elongated structure from a patient.

2. Background Information

A variety of medical treatments and surgical methods entail implanting an elongated structure in the body of a human or veterinary patient. Examples of such elongated structures include catheters, sheaths and cardiac electrical leads (such as pacemaker leads and defibrillator leads), and a variety of other devices. Over time, it can become necessary or desirable to remove such an elongated structure from the body of the patient. However, problems can be encountered in attempting removal of an elongated structure implanted in biological tissue.

For example, a heart pacemaker is typically implanted in a subcutaneous tissue pocket in the chest wall of a patient, and a pacemaker lead positioned in the vascular system of the patient, extending from the pacemaker and through a vein into a chamber of the patient's heart. The pacemaker lead commonly includes a coiled structure such as an electrical wire coil for conducting electrical signals (such as stimulating and/or sensing signals) between the pacemaker and the heart. Defibrillator leads are generally similar and, like pacemaker leads, are located about the heart, but are affixed both internally and externally of the heart. Some leads include one or more coaxial or lateral helical wire coils having a hollow inner passageway that extends the entire length of the wire coil or coils. Other leads may be made with a cable without a hollow inner passageway. The wire coils are surrounded by an electrically insulating material such as a flexible tube, sheath or coating. The insulating material can be silicone or polyurethane, and serve simultaneously to protect the wire coils from body fluids and to insulate the wire coils from one another.

While cardiac electrical leads typically have a useful life of many years, over time pacemaker and defibrillator leads unfortunately become encapsulated by fibrotic tissue against the heart itself or the wall of the vein, or against other surrounding tissue. Encapsulation is especially encountered in areas where the velocity of the flow of blood is low. The fibrotic tissue is tough and makes it difficult to remove the lead from the area of the heart without causing trauma to the area. For example, when small diameter veins through which a pacemaker lead passes become occluded with fibrotic tissue, separating the lead from the vein can cause severe damage to the vein such as dissection or perforation of the vein. Furthermore, separation of the lead from the vein is usually not possible without restricting or containing movement of the lead, i.e., fixing the lead in position with respect to the patient, in particular, with respect to the patient's vein.

To avoid this and other possible complications, some useless pacemaker or other leads are simply left in the patient when the pacemaker or defibrillator is removed or replaced. However, such a practice can incur the risk of an undetected lead thrombosis, which can result in stroke, heart attack, or pulmonary embolism. Such a practice can also impair heart function, as plural leads can restrict the heart valves through which they pass.

There are of course many other reasons why removal of a useless lead is desirable. For example, if there are too many leads positioned in a vein, the vein can be obliterated. Multiple leads can be incompatible with one another, interfering with the pacing or defibrillating function. Of course, an inoperative lead can migrate during introduction of an adjacent second lead, and mechanically induce ventricular arrhythmia. Other potentially life-threatening complications can require the removal of the lead as well. For example, removal of an infected pacemaker lead is desirable, so as to avoid septicemia or endocarditis.

Surgical removal of a heart lead in such circumstances often involves open heart surgery, with its accompanying risks, complications and significant costs. A variety of successful methods and apparatus have been devised as alternatives to open heart surgery for heart lead removal. Several such successful methods and apparatus are described in related application Ser. No. 08/433,820, filed May 4, 1995, entitled “Device for Removing an Elongated Structure Implanted in Biological Tissue,” now U.S. Pat. No. 5,697,936, assigned to the assignee of the present invention. The related patent, U.S. Pat. No. 5,697,936 is a continuation-in-part of application Ser. No. 08/255,602, filed Jun. 8, 1994, entitled “Locally Flexible Dilator Sheath,” now U.S. Pat. No. 5,507,751; which was in turn a continuation-in-part of then-pending application Ser. No. 08/042,375, filed Apr. 2, 1993, entitled “Apparatus for Removing an Elongated Structure Implanted in Biological Tissue,” now U.S. Pat. No. 5,632,749; which was a divisional of then-pending application Ser. No. 07/691,706, filed Apr. 26, 1991, now U.S. Pat. No. 5,207,683, entitled “Apparatus for Removing an Elongated Structure Implanted in Biological Tissue;” which was a continuation-in-part of then-pending application Ser. No. 07/363,960, filed Jun. 9, 1989, now U.S. Pat. No. 4,943,289, entitled “Apparatus for Removing an Elongated Structure Implanted in Biological Tissue;” which was a continuation-in-part of then-pending application Ser. No. 07/347,217, filed May 3, 1989, now U.S. Pat. No. 5,011,482, entitled “Apparatus for Removing an Elongated Structure Implanted in Biological Tissue;” which was a continuation-in-part of then-pending application Ser. No. 07/298,100, filed Jan. 17, 1989, now U.S. Pat. No. 5,013,310, entitled “Method and Apparatus for Removing an Implanted Pacemaker Lead;” which was a continuation-in-part of then-pending application Ser. No. 07/269,771, filed Nov. 9, 1988, now U.S. Pat. No. 4,988,347, entitled “Method and Apparatus for Separating a Coiled Structure from Biological Tissue.” All of the aforementioned applications and issued patents are incorporated by reference as if fully set forth herein.

Related U.S. Pat. No. 5,697,936 discloses a device for removing from a patient a previously implanted elongated structure, the structure having an outside dimension, a free proximal end located either inside or outside the patient, and a distal end normally fixedly located within the patient. The device comprises a sheath member having an inside dimension greater than the outside dimension of the elongated structure, and a reversibly collapsible snare associated with the sheath member. The sheath member delivers the snare to one of the proximal and distal ends of the elongated structure. The snare is dimensioned to encircle the same one of the proximal and distal ends of the elongated structure, and comprises a coil, loop, cylindrical cage or coiled leaf spring. Additional improved embodiments of the reversibly collapsible snare and sheath member are desirable. For example, it would be desirable if a device for removing from a patient a previously implanted elongated structure comprised a gripping member having a structure that contracted onto the elongated structure when subjected to a longitudinal force. It would be desirable if the longitudinal force could be imparted either directly, for example by pulling a portion of the gripping member that extends outward beyond the proximal end of the elongated structure and outwardly of at least the vascular system of the patient, or indirectly, for example via a draw means that extends outside at least the vascular system of the patient. The gripping member can comprise a double helical structure, a spring structure, a multi-stranded woven or braided structure, a fenestrated structure, a polymeric tubular structure, an elastomeric tubular structure and/or a gripping knot. Also, it would be desirable if the gripping member was capable of self-tightening. It would also be desirable if the sheath member was readily removable after the gripping member was positioned on the elongated structure. In addition, it would be desirable if the sheath member provided a convenient packaging system that maintained the geometry of the gripping member prior to use. Further, it would be desirable if the device was adapted to be placed in difficult to reach areas within a patient.

### SUMMARY

In one aspect, the invention is a device for removing from a patient a previously implanted elongated structure; the elongated structure having an outside dimension, a proximal end and a distal end located within the patient; the device comprising: a sheath member having an inside dimension greater than the outside dimension of the elongated structure; a gripping member positioned about the sheath member, the gripping member having a proximal end, a distal end, a longitudinal passage extending between the proximal end and the distal end, the longitudinal passage dimensioned to encircle at least a portion of the elongated structure, and a proximal portion that extends outwardly beyond the proximal end of the elongated structure and outwardly of at least the vascular system of the patient, wherein the gripping member comprises a first configuration having a first diameter that is greater than the outside dimension of the elongated structure, and a second configuration having a second diameter that is about the same as the outside dimension of the elongated structure.

In another aspect, the invention is a device for removing from a patient a previously implanted elongated structure; the elongated structure having an outside dimension, a proximal end, and a distal end located within the patient; and the device comprising: a splittable sheath member having an inside dimension greater than the outside dimension of the elongated structure; and a gripping member positioned about the splittable sheath member, wherein the gripping member comprises a first configuration having a first diameter that is greater than the outside dimension of the elongated structure and a second configuration having a second diameter that is about the same as the outside dimension of the elongated structure.

In another aspect, the invention is a device for removing from a patient a previously implanted elongated structure; the elongated structure having an outside dimension, a proximal end, and a distal end located within the patient; and the device comprising: a sheath member having an inside dimension greater than the outside dimension of the elongated structure; a gripping member positioned about the sheath member and dimensioned to encircle at least a portion of the elongated structure; and a draw means connected to the gripping member and extending outwardly of the patient; wherein the sheath member is configured as a packaging system that maintains the geometry of the gripping member and the draw means prior to use.

In another aspect, the invention is a device for removing from a patient a previously implanted elongated structure; the elongated structure having an outside dimension, a proximal end, and a distal end located within the patient; and the device comprising: a sheath member having an inside dimension greater than the outside dimension of the elongated structure; and a gripping member comprising a gripping knot positioned about the sheath member and dimensioned to encircle at least a portion of the elongated structure, wherein the gripping member comprises a first configuration having a first diameter that is greater than the outside dimension of the elongated structure and a second configuration having a second diameter that is about the same as the outside dimension of the elongated structure.

In yet another aspect, the invention is a device for removing from a patient a previously implanted elongated structure; the elongated structure having an outside dimension, a proximal end, and a distal end located within the patient; and the device comprising: a sheath member having an inside dimension greater than the outside dimension of the elongated structure; and a self-tightening gripping member positioned about the sheath member and dimensioned to encircle a portion of the elongated structure; wherein the self-tightening gripping member comprises a first configuration having a first diameter that is greater than the outside dimension of the elongated structure, and a second configuration having a second diameter that is abut he same as the outside dimension of the elongated structure.

In other aspects of the invention, the gripping member can comprise a double helix of opposite hands, a helical spring, a braided structure, a pattern of fenestrations, a woven structure, a polymeric tubular structure or an elastomeric tubular structure. Optionally, the gripping member can comprise a surface that has been mechanically or chemically treated to provide a frictional force that enhances gripping.

In other aspects of the invention, the sheath member can be splittable. Optionally, the splittable sheath member can comprise a groove or a score or a pre-slit end or a weakened area to facilitate splitting.

In another aspect of the invention, the device can also comprise a deployment structure to deliver the device to difficult to reach areas. Optionally, the deployment structure can be splittable. In other aspects, the deployment structure can comprise a groove, a score, a weakened area or pre-slit end to facilitate splitting.

In yet another aspect of the invention, the device can also comprise a grasping means attached to the distal end of gripping member to facilitate the placement and the locking of the gripping member onto the elongated structure.

### BRIEF DESCRIPTION OF THE DRAWINGS

The present invention will be more fully appreciated as the same becomes better understood from the following detailed description when considered in connection with the accompanying drawings, in which like reference numerals and characters designate like or corresponding parts through the several views.

FIGS. 1-1B are schematic views demonstrating a preferred embodiment of the present invention;

FIG. 1C is a schematic view of another preferred embodiment of the present invention;

FIGS. 2-2B are schematic views of another preferred embodiment of the present invention;

FIG. 2C is a schematic view of another preferred embodiment of the present invention;

FIGS. 3-3A are schematic views demonstrating another preferred embodiment of the present invention;

FIGS. 4-4B are schematic views of an alternate preferred embodiment of the present invention;

FIGS. 4C-4D are schematic views of an alternate preferred embodiment of the present invention;

FIGS. 5-5B are schematic views of another preferred embodiment of the present invention;

FIGS. 5C-5D are schematic views of another preferred embodiment of the present invention;

FIGS. 6-6B are schematic views of another preferred embodiment of the present invention;

FIG. 7 is a schematic representation of another preferred embodiment of the present invention in its packaged configuration;

FIG. 8 is an enlarged detail view of a portion of the preferred embodiment illustrated in FIG. 7;

FIG. 9 is a schematic view of the preferred embodiment illustrated in FIGS. 1-1B as it arrives from the manufacturer in its packaged configuration;

FIG. 10 is an enlarged detail view of a portion of the preferred embodiment illustrated in FIG. 9;

FIGS. 11-11A are schematic views of another preferred embodiment of the present invention;

FIGS. 12-12A are partial cross-sectional views of another preferred embodiment of the present invention during use;

FIG. 13 is a partial schematic view of another preferred embodiment of the present invention;

FIG. 14 is a partial schematic view of another preferred embodiment of the present invention;

FIG. 15 is a partial schematic view of another preferred embodiment of the present invention; and

FIG. 16 is a partial schematic view of another preferred embodiment of the present invention.

### DETAILED DESCRIPTION OF THE PREFERRED EMBODIMENTS

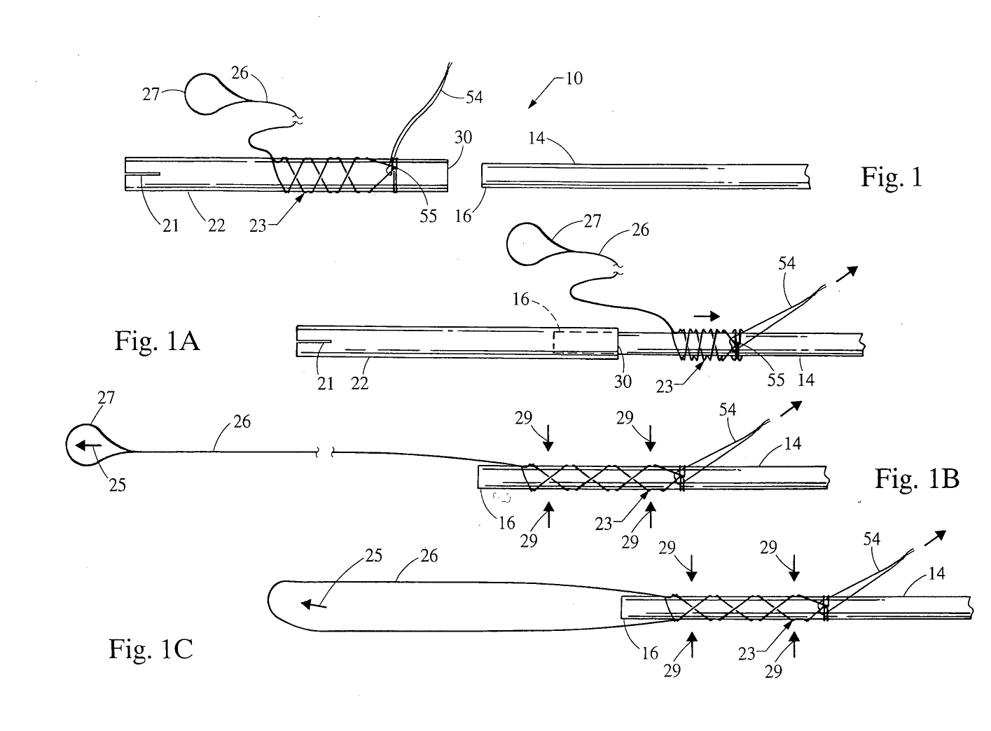

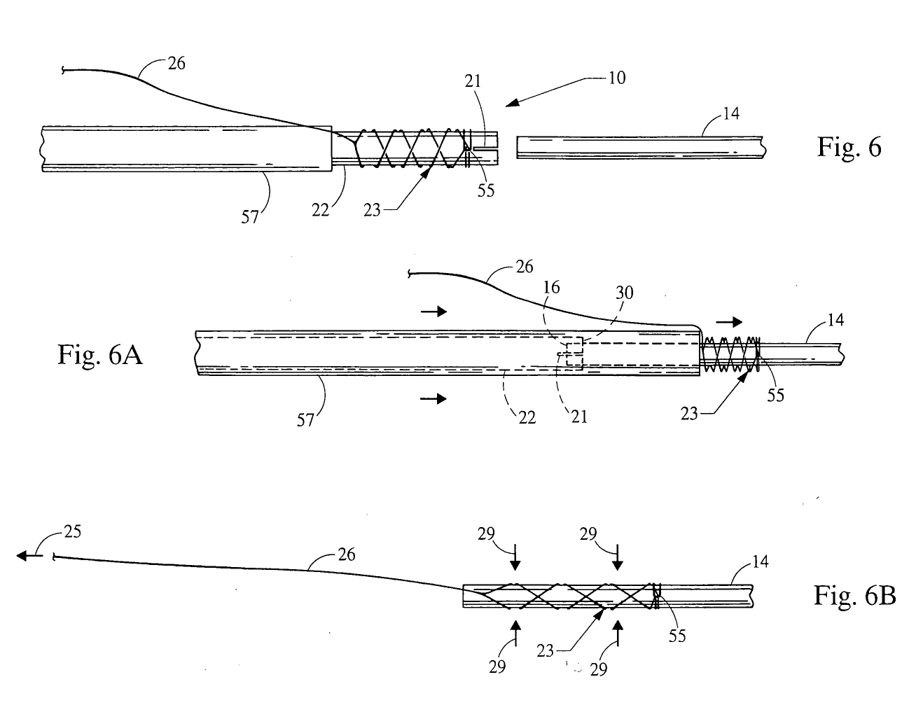

With reference first to FIGS. 1-16, a device 10 for removing or extracting a previously implanted elongated structure 14 from a patient is shown. The elongated structure 14 to be removed has an outside dimension or diameter, a proximal end 16 and a distal end located within the patient. The distal end of the elongated structure 14 may be located within or outside the vascular system of the patient. For example, when the elongated structure 14 is the cardiac pacemaker lead, the distal end will be located within the vascular system of the patient, and in particular, within a chamber of the patient's heart (such as in an atrium or ventricle of the heart). Alternatively, when the elongated structure 14 is a defibrillator lead, the distal end will be located either in or about the heart of the patient. The distal ends of other types of elongated structures 14 may not be and need not be near the heart at all; the device 10 will still be useful for removing them. The proximal end 16 of the elongated structure 14 may be located within or outside the vascular system of the patient and optionally outside the patient. While the device 10 of the present invention is particularly useful for removing pacemaker leads, it is also useful for removing other implanted, elongated bodies. Such bodies can be defibrillator leads or other cardiac electrical leads, catheters, sheaths, cannulae or the like.

The device 10 of the present invention is adapted to engage the elongated structure 14 to serve several different functions. The device 10 serves to fix the position of the structure 14 with respect to the patient to replace the current practice of manually applying a ligature around the proximal segment of the elongated structure 14 to be removed, such as a cardiac lead, into which a locking stylet has been placed. The device 10 can also remove an elongated structure 14 having a solid structure with no hollow inner passage. In addition, the device 10 can provide a cable length extension to the elongated structure 14 over which a coring cannula or sheath (such as a dilator sheath) can be introduced.

With reference to FIGS. 1-11A and 13-16, the device 10 comprises a sheath member 22 and a gripping member 23 associated with the sheath member 22. The sheath member 22 has an inside dimension or diameter greater than the outside dimension or diameter of the elongated structure 14, such that the sheath member 22 can be fit over at least the proximal end 16 of the elongated structure 14.

The gripping member 23 is positioned about, and carried by, the sheath member 22. The gripping member 23 has a proximal end and a distal end and a longitudinal passage extending between the proximal end and the distal end. The gripping member 23 is configured to engage and form a grip with the exterior surface of the elongated structure 14. The cross-section of the longitudinal passage may be uniform or varied to encourage gripping about the elongated structure. The gripping member 23 has a first configuration in which it is positioned over the exterior surface of the elongated structure 14 and a second configuration in which it engages the elongated structure 14. This engagement of the gripping member 23 to the elongated structure 14 forms a connection or “lock” to the elongated structure 14. Thus, the gripping member 23 has a narrower maximum diameter in the second configuration than in the first configuration. More particularly, the gripping member 23 is adjustable between the maximum diameter of the first configuration and the narrow diameter of the second configurations. Depending on the application, the gripping member 23 can be constructed to engage the elongated structure 14 in a localized area or substantially along the entire length of the elongated structure 14. More particularly, a gripping member 23 constructed to engage a localized area provides the advantage of removing an elongated structure 14 from an area of limited access. Alternatively, a gripping member 23 constructed to engage along substantially the entire length of the structure 14 provides the advantage of distributing the tractional force along the length of the structure 14, and thus reduces the risk of distorting or breaking the structure 14 during the removal process.

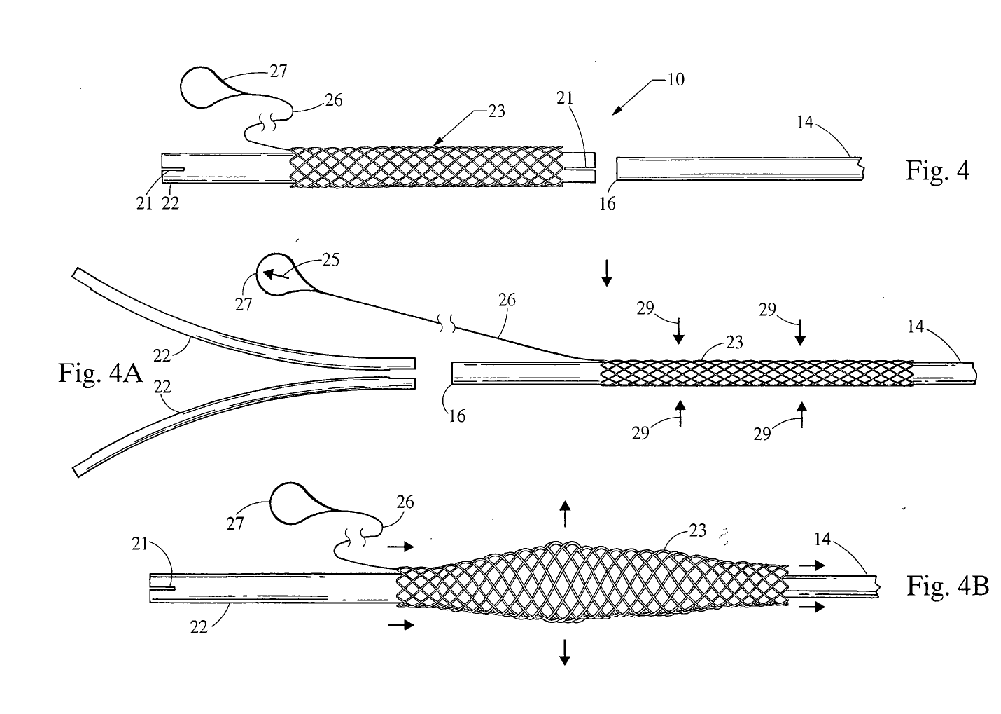

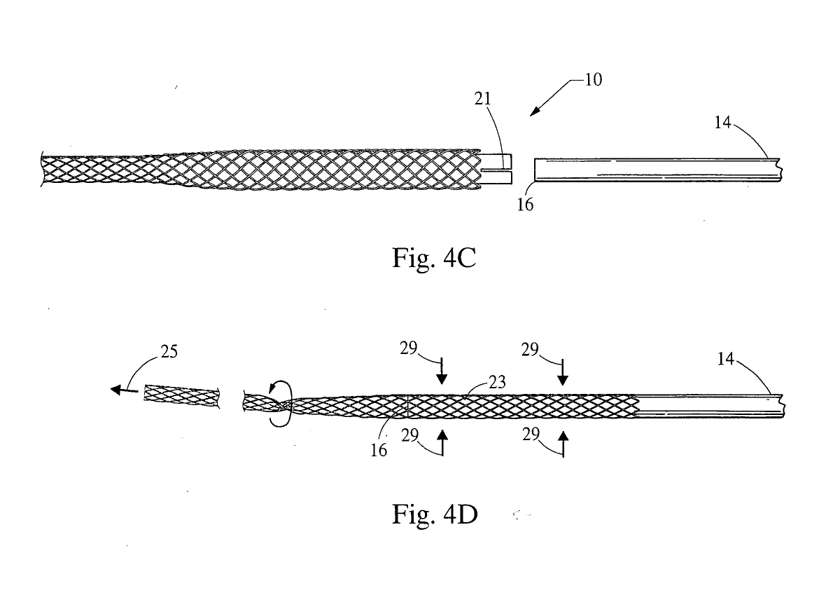

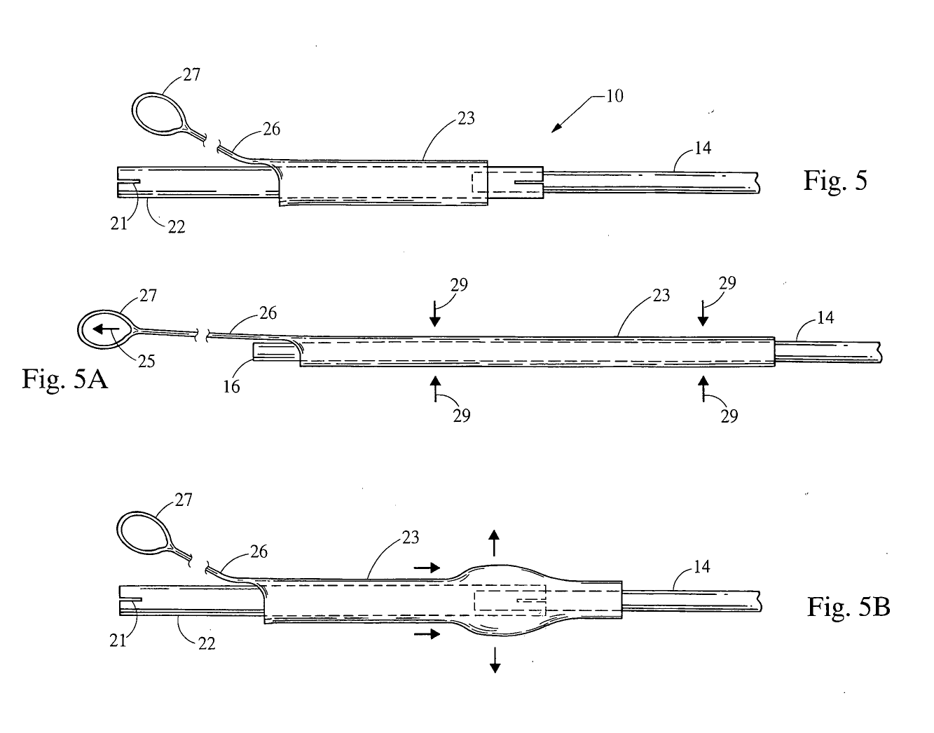

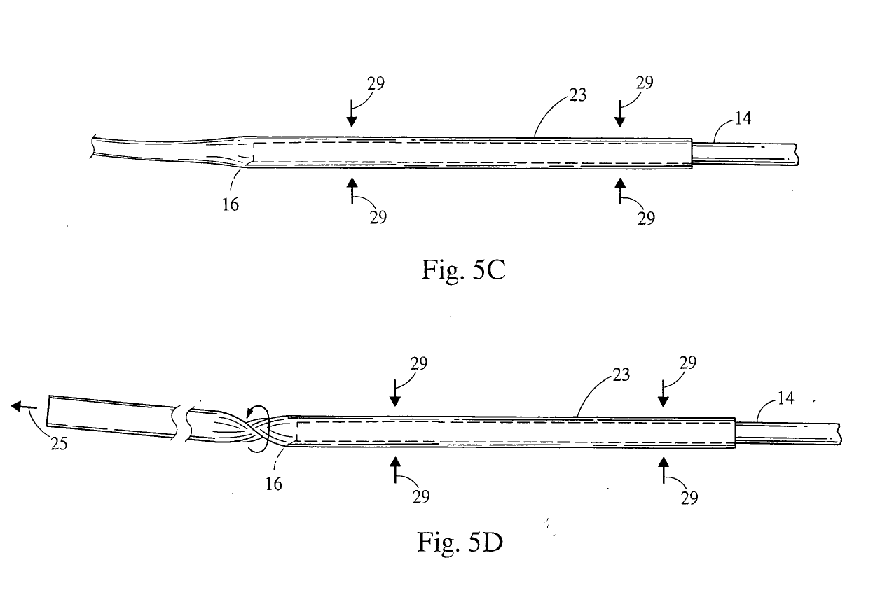

The gripping member 23 can comprise any structure that contracts about the elongated structure 14 when subjected to a first longitudinal force (e.g. tractional force shown by arrow 25). The resultant contractile force (shown by arrows 29) creates a sufficient longitudinal frictional force to “lock” the gripping member 23 to the elongated structure 14 to be removed. The locking of gripping member 23 to the elongated structure 14 is reversible by applying a second longitudinal force in a direction opposite to the first longitudinal force. The longitudinal forces that cause the gripping member 23 to contract or expand about the elongated structure 14 may be applied directly to the gripping member 23, as shown for example in FIGS. 4B-4D and 5B-5D, or may be applied indirectly, for example via a draw means 26 as shown in FIGS. 1-1C, 2-2C, 3-3A, 4-A, 5-5A, 6B and as explained in more detail below. With respect to the embodiments in which the longitudinal force is applied directly to the gripping member, the gripping member 23 is positioned on the elongated structure 14 and a portion of the gripping member 23 extends outwardly beyond the proximal end 16 of the elongated member 14 and outside the vascular system of the patient, and optionally outside the patient. FIGS. 4C-4D and 5C-5D shown that as a longitudinal force is applied to the outwardly extending portion of the gripping member 23 (e.g. tractional force, arrow 25), the gripping member 23 contracts (arrows 29) about the elongated structure 14. Optionally, the user may also simultaneously twist the gripping member 23 to cinch the gripping member 23 about the proximal end 16 of the elongated structure 14 as the gripping member is pulled proximally as shown in FIGS. 4D and 5D. Conversely, a longitudinal force applied directly to the gripping member 23 in the opposite direction (e.g. distally) causes the gripping member 23 to expand as shown in FIGS. 4B and 5B.

With respect to the embodiments in which the longitudinal force is applied indirectly to the gripping member, shown in FIGS. 1-1C and 2-12A, the device 10 further comprises a draw means 26 connected to the gripping member 23 through which a longitudinal force can be applied. Optionally, the connection between draw means 26 and gripping member 23 may comprise multiple points of attachment as shown in FIG. 16. Draw means 26 extends outwardly of the vascular system of the patient, and optionally outwardly from the patient. As shown in FIGS. 1-5B, the draw means 26 may also include a proximal loop 27 that acts as a graspable handle. As seen in FIGS. 1B, 2B-2C, 3A, 4B, 5B and 6B, draw means 26 is moveable in a first direction (e.g. proximally) to exert a longitudinal force (e.g. tractional force, shown by arrow 25) that acts to collapse the gripping member 23 (indicated by arrows 29) about the encircled portion of the elongated structure 14. FIG. 1C depicts a non-limiting alternate embodiment of draw means 26. The tractional force can be delivered by pulling on proximal loop 27 as shown in FIGS. 1B, 2B-2C, 3A, 4B and 5B. Optionally, the draw means 26 is sufficiently rigid to be moveable in the direction opposite to the first direction (e.g. distally) so as to expand the gripping member 23 and free it from the encircled structure 14. This ensures that the engagement of the gripping member 23 with the elongated structure 14 is reversible. Thus, the gripping member 23 can be disengaged from the elongated structure 14 and readily removed from the patient should termination of the removal procedure be necessary, for example, in case ventricular arrhythmia or another complication develops.

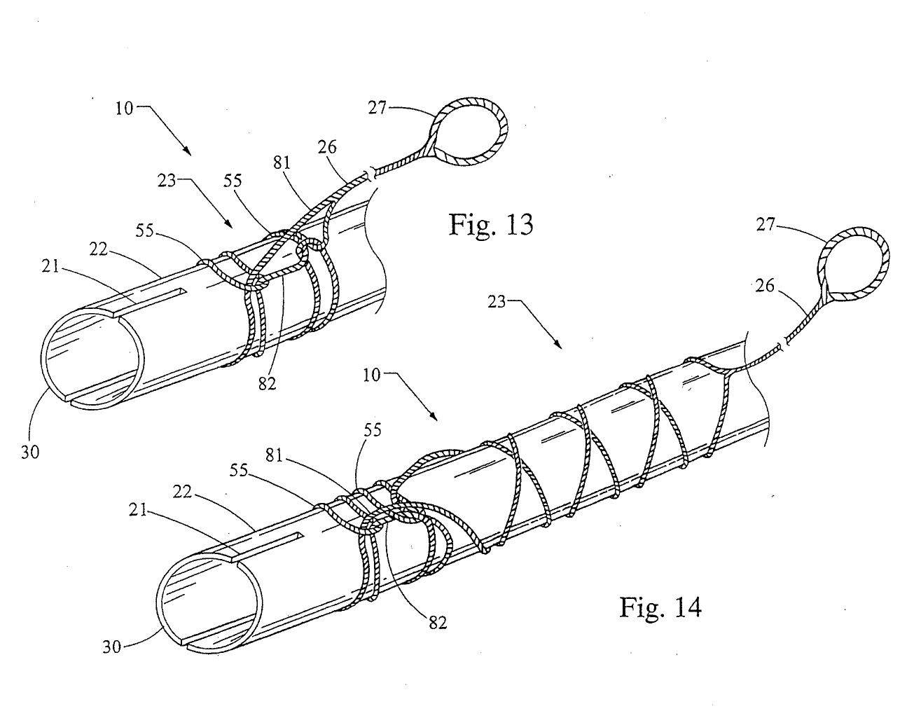

Optionally, the gripping member 23 may further comprise one or more gripping knots 55. For example, FIGS. 1-1C, 6-6B, and 7-10 show a gripping knot 55 positioned at the distal end of the gripping member 23. Alternatively, one or more gripping knots may be positioned along the length of the gripping member. For example, FIG. 13 shows a gripping member 23 comprising multiple gripping knots 55 and FIG. 14 shows multiple gripping knots 55 positioned at the distal end of the gripping member 23 in combination with a criss-crossed double helix. Gripping knot 55 acts as an anchor that provides a frictional force to enhance the grip of gripping member 23 about elongated member 14 during the removal process. Although not specifically shown in each figure, any embodiment of the gripping member shown and described herein may comprise one or more gripping knots along its length.

Optionally, the gripping member 23 may further comprise a surface that has been mechanically or chemically treated to enhance the gripping of gripping member 23 to the elongated structure 14. For example, the surface can comprise a texture or the surface can be coated with particles or adhesives. Alternatively, the material used to form the gripping member 23 can be impregnated with particles or adhesive to enhance gripping.

Optionally, device 10 can also comprise a grasping means, such as a suture loop 54, attached to the gripping member 23 as shown in FIGS. 1-1B, 9 and 10. Although not specifically shown in each figure, suture loop 54 may be attached to any embodiment of the gripping member shown or described herein. Suture loop 54 facilitates the placement of the device 10 and the locking of gripping means 23 onto the elongated structure 14 to be removed. As shown in FIG. 1A, the suture 54 can be pulled distally as gripping member 23 is pushed distally to position the grasping member 23 onto the elongated structure 14 to be moved. As shown in FIG. 1B, suture 54 can be pulled distally as draw means 26 is pulled proximally causing gripping member 23 to contract and thereby lock gripping means 23 onto the structure 14. After the gripping means 23 is secured to the elongated structure 14, suture loop 54 can be cut and removed. By way of a non-limiting example, a suitable material for grasping means is size 2-0, braided black, nylon, suture thread.

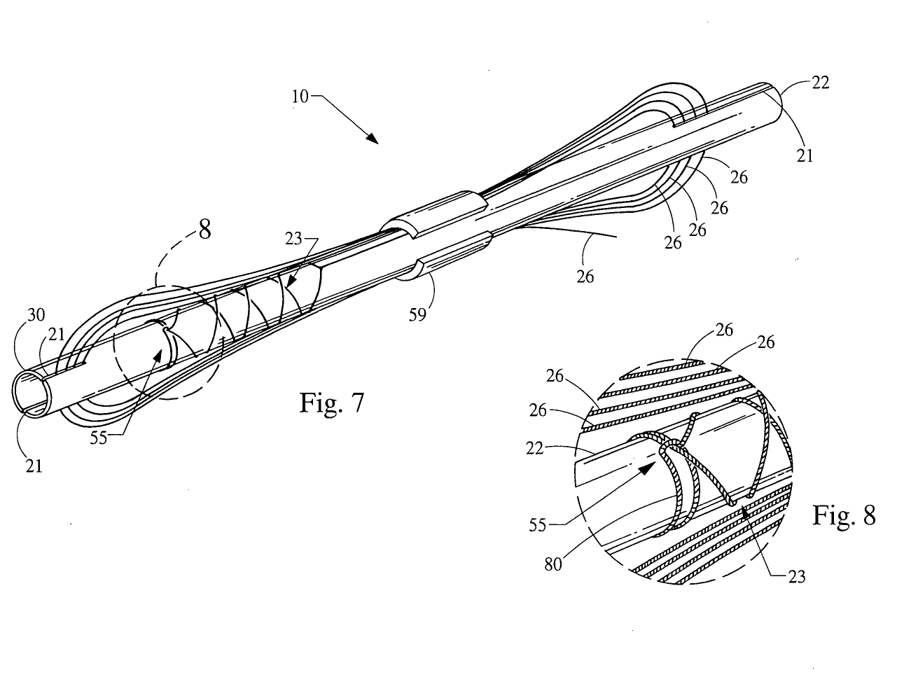

As shown in FIGS. 1-16, several alternative embodiments have been contemplated for gripping means 23. For example, the gripping member 23 can be configured as a crisscrossed (or intertwined) double helix as shown in FIGS. 1-1C, 6-10 and 14. By stretching the double helix longitudinally, locking is effected. Such movement is easily accomplished by pulling draw means 26 or proximal loop 27 in the proximal direction. The crisscrossed double helix gripping member 23 can be formed with a gripping knot or without a gripping knot. Any combination of criss-crossed helices and/or gripping knot is contemplated. The criss-crossed helical gripping member 23 can be formed from any suitable material known in the art including, but not limited to, cable, wire, ribbon or tubing. For example, FIGS. 1-1C, 6-10 and 14 illustrate a criss-crossed double helical gripping member 23 formed from a cable. One suitable non-limiting example is a 304 stainless steel cable having a 3×7 construction and a 0.009″ outer diameter. The cable is twined into a double helical configuration at the distal portion of the device 10. As best seen in FIGS. 7-10, by way of a non-limiting example, the gripping member 23 may be formed by folding a length of stainless steel cable in half to form a loop or bight 80, the loop or bight 80 is wrapped about the sheath member 22 and the two ends of the cable 81, 82 are passed through the loop or bight 80 to form a gripping knot 55 about the sheath member 22. Gripping knot 55 provides a frictional force to enhance the grip of gripping member 23 about elongated member 14 during the removal process. The cable ends 81, 82 are then twined into a double helical configuration to form gripping member 23. The cable ends 81, 82 can be secured together by any means known in the art to form draw means 26, including, but not limited to, soldering, heat fusing, adhesive bonding, chemical bonding or mechanical coupling such as a cannulae. Alternatively, as shown in FIG. 14, additional gripping knots 55 may be formed after the first gripping 55 knot by forming a second loop or bight 80 with one of the cable ends 81, 82 passed through the first loop or bight 80, wrapping the second loop or bight 80 about the sheath member 22 and passing the cable end through the second loop or bight 80 to form a second gripping knot 55. The cable ends 81, 82 are then twined into a double helical configuration to form gripping member 23.

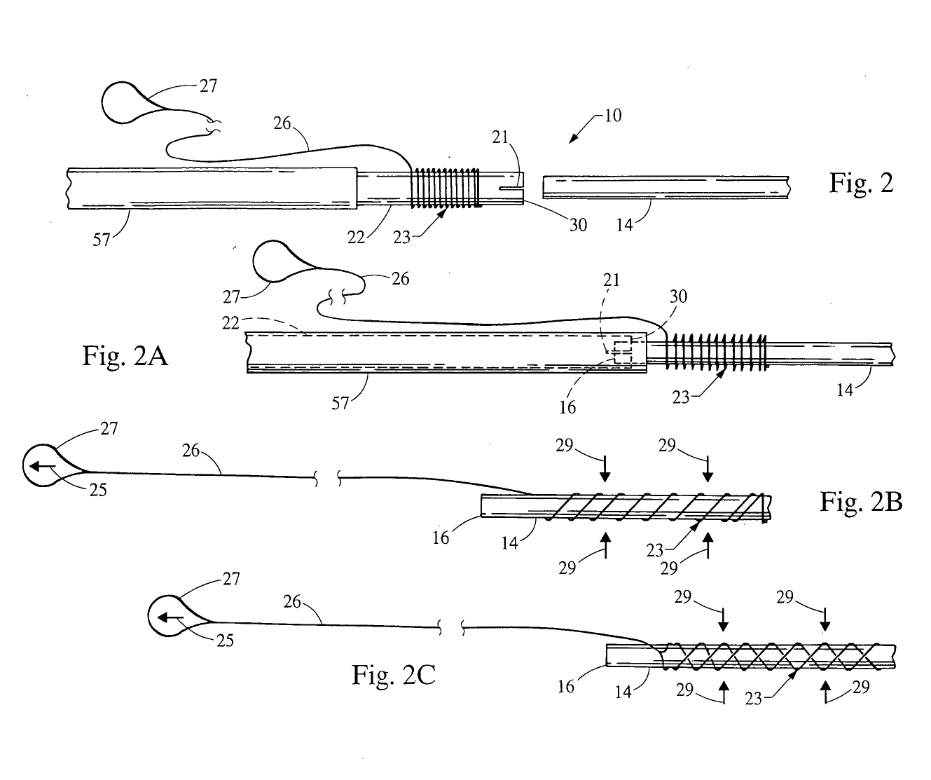

Alternatively, as shown in FIGS. 2-2C, the gripping means 23 can comprise a helical spring. The helical spring gripping member 23 can be formed from any suitable material known in the art including, but not limited to, cable, wire, ribbon or tubing. FIG. 2 shows a gripping means 23 comprising a helical spring positioned on a distal portion of the device 10. As can be seen in FIG. 2A, the diameter of the helical spring positioned on the elongated structure 14 is substantially the same as the diameter of the helical spring mounted on the sheath member 22. However, as shown in FIG. 2B, as a longitudinal force (e.g. tractional force shown by arrow 25) is applied to gripping member 23, e.g. by pulling draw means 26 or by pulling a portion of the gripping member 23 that extends outside the vascular system of a patient (not shown), the diameter of the helical coil contracts to lock onto the elongated structure 14. FIG. 2C shows that at least two helical springs of opposite hands can be used to form gripping member 23. Using two helical springs of opposite hands prevents the elongated structure from twisting and un-twisting during the removal process.

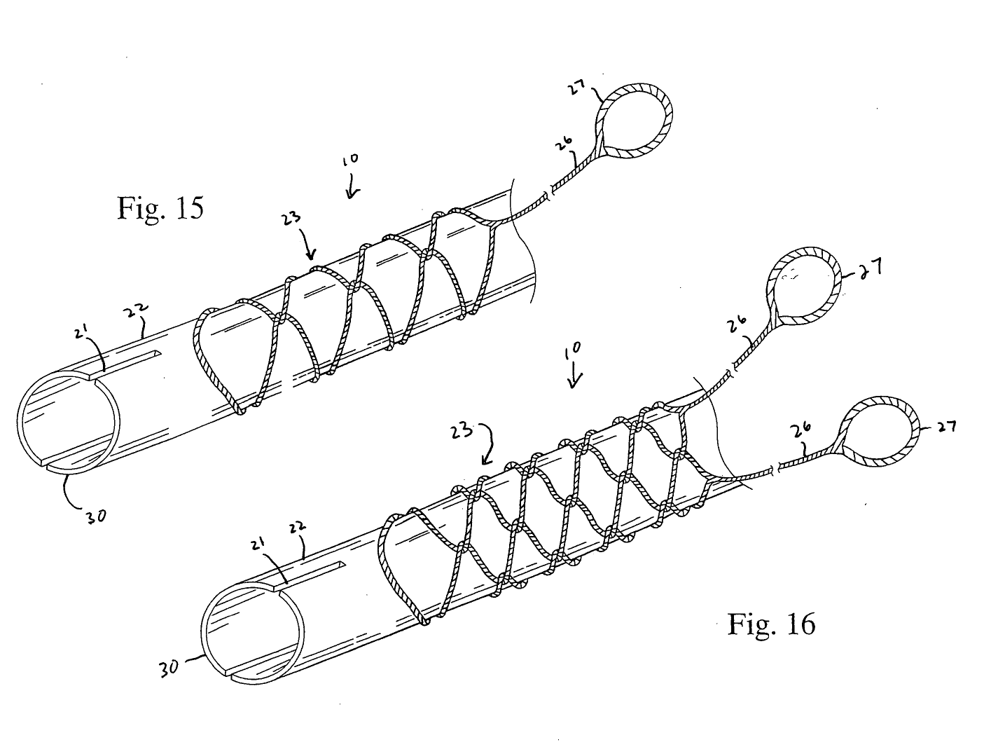

Alternatively, the gripping member 23 can comprise any braided or woven structure that contracts when a longitudinal force is exerted upon it. The braided or woven gripping member 23 can be formed from any suitable material known in the art including, but not limited to, cable, wire, ribbon or tubing. Typically, a braided structure comprises three or more strands or filaments. A woven structure may comprise a single or multiple strands or filaments. By way of a non-limiting example, FIGS. 4-4C show a gripping member 23 comprising a multi-stranded braided structure. More particularly, the gripping member 23 comprises a braided sheath of flat wires (or metal ribbon), which have a rectangular cross-section. The flat wires of the braided sheath comprise 304 stainless steel, for example. As with the previously described embodiments, the diameter of the braided sheath contracts when a longitudinal force in the proximal direction is applied either indirectly (FIG. 4A) or directly (FIG. 4D). Conversely, pushing the draw means 26 distally reverses the contraction of gripping member 23 and releases it from the elongated structure 14 as shown in FIG. 4AB. Thus, as the physician pushes the gripping member 23 distally off the sheath member 22, the diameter of the gripping member 23 expands. This expansion facilitates the positioning of the gripping member 23 onto the elongated structure 14. By way of additional non-limiting examples, FIG. 15 illustrates an alternate weave configuration and FIG. 16 illustrates an alternate double weave configuration. Any weave and/or gripping knot combination is contemplated.

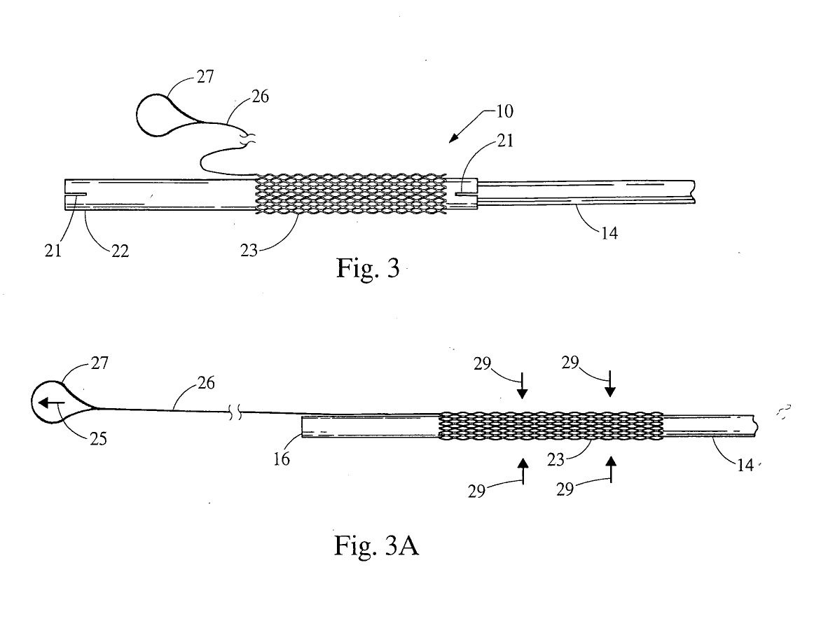

Alternatively, as shown in FIGS. 3-3A, the gripping member 23 can comprise a pattern of fenestrations that effects a contraction of the gripping member 23 onto the elongated structure 14 upon exertion of a longitudinal force to the gripping member 23. The gripping member can be fabricated as a single piece of material by stamping or cutting (e.g. with a laser), fabricating a mold or some similar method of producing a unitary gripping member. A non-limiting example of a fenestration pattern is shown in FIGS. 3-3A.

Alternatively, the gripping member 23 can comprise any polymeric or elastomeric structure that effects a contraction of the structure about the target lead upon exertion of a longitudinal force upon the structure. For example, as shown in FIGS. 5-5B, the gripping member 23 can comprise a polymeric or elastomeric tubular member. The polymeric or elastomeric tubular member can be molecularly orientated to effect a diametrical contraction of the tubular member upon the exertion of a proximal longitudinal force as shown in FIGS. 5 and 5B. Conversely, the application of a longitudinal force in the opposite direction (distally) reverses the contraction of the tubular member about the elongated structure. As shown in FIG. 5A, the molecular orientation can be designed such that as the physician pushes the gripping member 23 off the sheath member 22, the diameter of the gripping member 23 expands to facilitate the placement of the device 10 on the elongated structure 14.

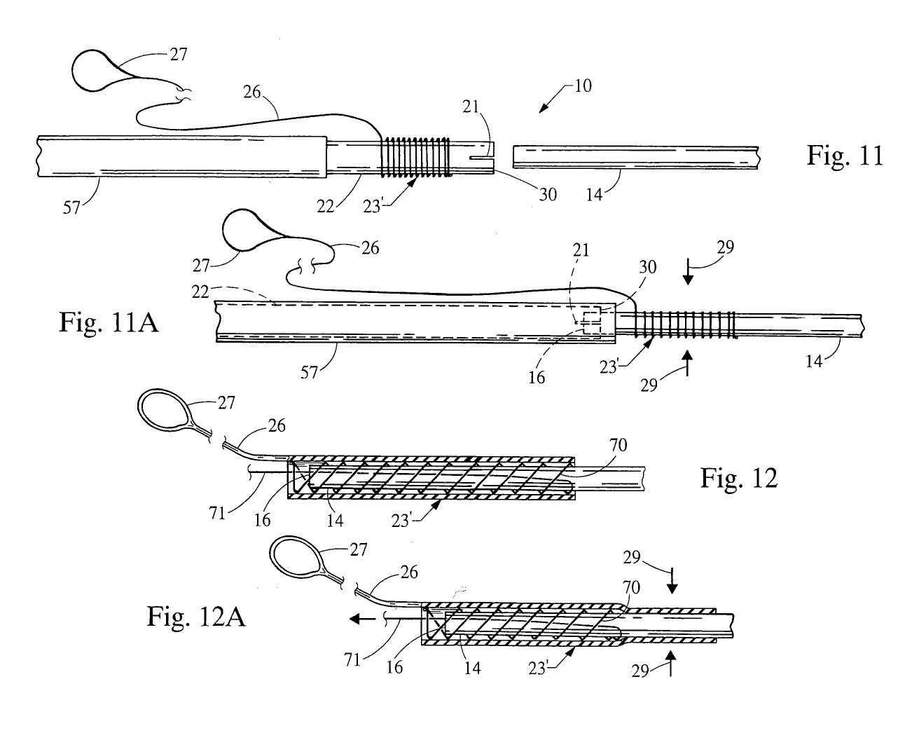

Alternatively, all of the previously disclosed embodiments of the gripping member 23 can be self-tightening, i.e., the gripping member 23 can be pre-tensioned or comprise spring-loaded properties. For example, as shown in FIGS. 11-12A, in the self-tightening embodiment, the locking of the device 10 to the elongated structure 14 results from the pre-tensioned or spring loaded properties of the self-tightening gripping member 23′ rather than a longitudinal force applied via the draw means 26. The self-tightening gripping member 23′ is held in an “open” or “expanded” position until it is positioned onto the elongated structure 14. FIG. 11 shows the self-tightening gripping member 23′ held in the open or expanded configuration by the sheath member 22. Once the self-tightening gripping member 23′ is pushed off the sheath member 22 and slid onto the elongated structure 14, the locking of the device 10 to the elongated structure 14 becomes automatic. FIG. 11A shows the self-tightening gripping member 23′ contracted about the elongated structure 14 to be removed. Other structures can be used to maintain the self-tightening gripping member 23′ in the open configuration. By way of a non-limiting example, FIGS. 12-12A show a self-tightening gripping member 23′ held open by a helical dispensing coil 70 formed from wire 71. As shown in FIG. 12A, pulling wire 71 proximally unwinds the helical dispensing coil 70 and removes the support holding the self-tightening gripping member 23′ in the open configuration. As the helical dispensing coil 70 unwinds the self-tightening gripping member 23′ contracts onto the elongated structure 14 as shown in FIG. 12A.

The self-tightening feature of the self-tightening gripping member 23′ can result from the structural configuration of the gripping member 23′, or can be imparted to the gripping member 23′ by another structure placed over, or placed with, the gripping member 23′. One non-limiting example of a structure that is placed over the gripping member 23 to create the pre-tensioning is a tensioned sheath.

In accordance with the invention, both the gripping member and the draw means may be made of various materials, which can differ from each other, and can have different sizes and strengths. The gripping member and the draw means can be formed from any suitable material known in the art including, but not limited to, cable, wire, ribbon or tubing. Although the embodiments shown depict either round or flat wires and cables, other types of wire and cable, e.g., square, triangular or oval, may be used. Indeed, square, triangular and oval wires and cable provide an intermittent, non-uniform contact between the gripping member wire or cable and the elongated structure, which is believed to cause the gripping member wire or cable to twist during the removal process and result in enhanced gripping between the gripping member and the elongated structure. Suitable types of materials include metals, metal alloys, titanium, titanium alloy, nickel titanium alloy known to be shape-memory metals which are sold and manufactured under the trademark “NITINOL,” an alloy of tantalum or any other biocompatible material with elasticity may in certain circumstances be employed to advantage. When nickel titanium alloy is used, the material is operating in the linear portion of the stress/strain curve of the alloy, though it is possible to employ material operating in the super-elastic region while obtaining benefits of the invention. Likewise, thermally responsive shape memory metal can be employed with the geometric and spatial constraints provided by the invention. Alternatively, materials formed from various polymers, elastomers and plastics can be used. It is also anticipated that new materials, as they are developed, will be useful.

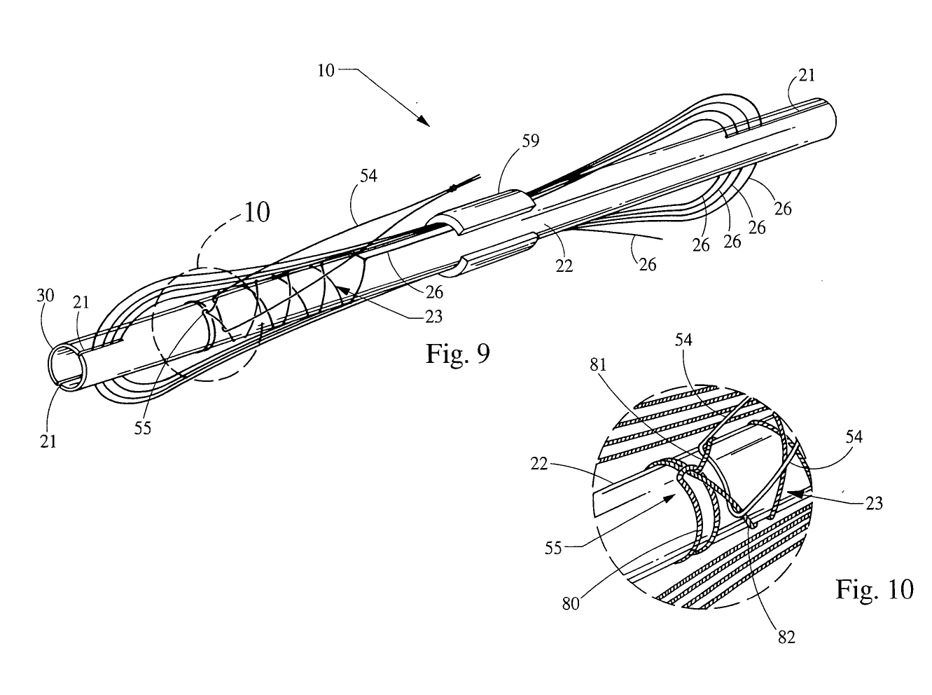

Several alternative embodiments have been contemplated for sheath member 22. The sheath member 22 serves as a delivery system that delivers the gripping member 23 to the proximal end 16 of the elongated structure 14 as shown in FIGS. 1-1A, 2-2A, 3, 4-4A, 5-5A and 6-6A. Thus, the sheath member 22 has an inside dimension or diameter greater than the outside dimension or diameter of the elongated structure 14 so that the sheath member 22 can be fit over the proximal end 16 of the structure 14. The sheath member 22 carries the gripping member 23, which can be affixed to the sheath member 22 as shown in FIGS. 7 and 9. Optionally, to assist in the ready removal of the elongated structure 14, the sheath member 22 can be constructed of a splittable material, i.e. a material that readily tears in a longitudinal direction along the length of the sheath. A non-limiting example of a splittable material is a molecularly oriented (non-isotropic) polytetrafluoroethylene (PTFE) such as that used in the PEEL-AWAY™ Introducer Sheath (Cook Incorporated, Bloomington, Ind.). Alternatively, the sheath member 22 can comprise a groove, pre-score or weakened area to facilitate the splitting of the sheath member 22 by the physician. In yet another alternative, the sheath member 22 can comprise one or more pre-slit ends 21 to initiate splitting of the sheath member 22 as best shown in FIGS. 7 and 9.

Optionally, as shown in FIGS. 7-10, the sheath member 22 also serves as a convenient packaging system that maintains the geometry of the gripping member 23 and the draw means 26 prior to use. For instance, the draw means 26 can be wound about the sheath member 22 to prevent the device from becoming entangled during transport. Optionally, the draw means 26 can be wound about the length of the sheath member 23 as shown in FIGS. 7 and 9. The draw means 26 can be wrapped through the pre-split ends 21 of the sheath member 22 to help maintain its wrapped configuration. Optionally, a removable, flexible band 59 can be placed about the wrapped draw means 26 and the sheath member 22 to maintain the packaged configuration.

As shown in FIGS. 2-2A, 6-6A and 11-11A, the device 10 can further comprise a deployment structure 57 to deliver the device 10 to difficult to reach areas, such as navigating the gripping member 23 through the vascular system of a patient and positioning the gripping member 23 proximal to the heart by way of a non-limiting example. Deployment structure 57 comprises an inside dimension or diameter greater than the outside dimension or diameter of the sheath member 22 so that the deployment structure 57 can be mounted for movement over sheath member 22. Once the device 10 has been positioned on the proximal end 16 of the elongated structure 14, the deployment device 57 can be pushed distally to position the gripping member 23 onto the elongated structure 14 as shown in FIGS. 2-2A, 6-6A and 12-12A. Optionally, the deployment structure 57 can be modified for ready removal in a similar manner as sheath member 22. In other words, deployment structure 57 can be constructed of a splittable material, can comprise a groove, pre-score or weakened area, or can comprise one or more pre-slit ends to facilitate the splitting of the deployment structure 57 by the physician. Although not specifically shown in each figure, the deployment structure 57 can be used with any of the disclosed embodiments of gripping member 23.

Use of the device 10 for fixing the position of the elongated structure 14 and freeing the structure 14 of any encapsulating tissue can be readily understood. The distal end of the sheath member 22 is positioned over the proximal end 16 of the elongated structure 14 as shown in FIGS. 1-1A, 2-2A, 3, 5, 6-6A and 11-11A.

Next, the gripping member 23 is positioned about at least a portion of the elongated structure 14. Depending on the length of the gripping member 23, the gripping member 23 can be placed along substantially the entire length of the elongated structure 14 or at any position along the length of the structure 14. The gripping member 23 can be manually positioned onto the structure 14 to be removed as shown in FIGS. 1-1A. For example, the physician pushes the gripping member 23 off the sheath member 22 and onto and along a portion of the elongated structure 14. Optionally, a grasping means comprising a suture 54 can be used to facilitate the positioning of gripping member 23 about the elongated structure. FIG. 1A illustrates suture 54 pulled distally as the gripping member 23 is pushed distally. Optionally, a deployment structure 57 can be used to push the gripping member 23, 23′ onto and along the structure 14 as shown in FIGS. 2-2A, 6-6A and 11-11A.

Next, the sheath member 22 is withdrawn. The sheath member 22 can be manually pulled off the proximal end of the elongated structure 14. Alternatively, if the sheath member 22 is splittable, it can be peeled away as shown in FIG. 4A.

After the sheath member 22 is removed, the gripping member 23 is collapsed onto a portion of the elongated structure 14. The locking of the gripping member 23 about a portion of the elongated structure 14 (indicated by arrows 29) can be accomplished by pulling on the draw means 26 (indicated by arrow 25) as shown in FIGS. 1-4A, 5-5A and 6-6B. In addition, FIG. 1B shows the optional use of a grasping means comprising a suture 54 to facilitate the locking of gripping member 23 to elongated structure 14. More particularly, the suture 54 is pulled distally as the draw means 26 is pulled proximally to lock the gripping member 23 about the elongated structure 14. Alternatively, gripping member 23 is collapsed about a portion of the elongated structure 14 by pulling the portion of the gripping member 23 that extends outwardly beyond the proximal end 16 of the elongated structure 14 and outwardly of the vascular system of the patient as shown in FIGS. 4C-4D and 5C-5D. In yet another alternative, if the self-tightening gripping member 23′ comprises a pre-tensioned structure, the gripping member 23 automatically contracts about the elongated structure 14 as shown in FIGS. 11-12A.

Once the gripping member 23, 23′ is locked to the elongated structure 14, a coring cannula or sheath, such as the dilator sheath, (not shown) then can be employed to separate the elongated structure from any encapsulating tissue, if necessary. The elongated structure 14 is then removed by pulling on the draw means 26 or proximal loop 27 (FIGS. 1B-1C, 2B-2C, 3A, 4A, 5A, 6B, 11A) or by pulling on the portion of the gripping member that extends outwardly beyond the proximal end of the elongated structure and outwardly of the vascular system of a patient (FIGS. 4D, 5D).

Use of the device 10 shown in FIGS. 1-12A for fixing the position of the elongated structure 14 and as an extension for introduction of the dilator sheath is also straightforward. Once the gripping member 23, 23′ has engaged the structure 14 to be removed, a dilator sheath can be advanced over the draw means 26, gripping means 23, 23′ and elongate structure 14 to separate it from any encapsulating tissue. The elongated structure 14 then can be removed from the patient by pulling on the proximal loop 27, the draw means 26 or the portion of the gripping member that extends outwardly beyond the proximal end of the elongated structure and outwardly of the vascular system of a patient.

Optionally, use of the device 10 to engage the proximal end 16 of the elongated structure 14 and free the structure 14 of any encapsulating tissue can be aided by fixing the position of the structure 14 with respect to the patient. If the structure 14 to be removed comprises a hollow longitudinal bore, a locking stylet can be engaged with the elongated structure for this purpose. For example, locking stylets of the type disclosed in the second through seventh patent applications and patents cross-referenced above, and expressly incorporated by reference herein, are particularly useful as the locking stylet. The locking stylet passes through the proximal end 16 of the elongated structure 14, and extends as far up the interior of the structure as possible. The distal end of the sheath member 22 is then positioned over the locking stylet and the proximal end 16 of the elongated structure 14. The gripping member 23, 23′ is attached to the elongated structure 14. A coring cannula or sheath, such as a dilator sheath, can then be used to cut encapsulating tissue away from the elongated structure 14. Finally, the elongated structure 14 can be removed by pulling proximal loop 27, draw means 26 or the portion of the gripping member that extends outwardly beyond the proximal end of the elongated structure and outwardly of the vascular system of a patient.

While the invention has been described with reference to several particular embodiments thereof, those skilled in the art will be able to make various modifications to the described apparatus and methods without departing from the true spirit and scope of the invention as hereinafter claimed.

1. A device for removing from a patient a previously implanted elongated structure; the elongated structure having an outside dimension, a proximal end, and a distal end located within the patient, the device comprising:
a) a sheath member having an inside dimension greater than the outside dimension of the elongated structure; and
b) a gripping member positioned about the sheath member, the gripping member having a proximal end, a distal end, a longitudinal passage extending between the proximal end and the distal end, the longitudinal passage dimensioned to encircle at least a portion of the elongated structure,
wherein the gripping member comprises a first configuration having a first diameter that is greater than the outside dimension of the elongated structure, and a second configuration having a second diameter that is substantially the same as the outside dimension of the elongated structure.

2. The device of claim 1, wherein the first configuration is dimensioned to encircle at least a portion of the elongated member and the second configuration is dimensioned to engage at least a portion of the elongated structure.

3. The device of claim 1, wherein the gripping member further comprises a gripping knot.

4. The device of claim 1, wherein the gripping member comprises a surface that has been mechanically or chemically treated to provide a frictional force between the gripping member and the elongated structure.

5. The device of claim 1, wherein the gripping member further comprises a proximal portion that extends outwardly beyond the proximal end of the elongated structure and outwardly of at least the vascular system of the patient.

6. The device of claim 1, further comprising a suture attached to the distal end of the gripping member.

7. The device of claim 1, wherein the sheath member is configured to deliver the gripping member to the proximal end of the elongated structure.

8. The device of claim 1, wherein the sheath member comprises a proximal end and a distal end, and at least one of said proximal and distal ends is pre-slit to facilitate the splitting of the sheath member.

9. The device of claim 1, wherein the sheath member comprises at least one of a groove, a score, and a weakened area.

10. The device of claim 1, wherein the sheath member comprises a splittable material.

11. The device of claim 10, wherein the splittable material comprises molecularly-oriented, non-isotropic polytetrafluoroethylene.

12. The device of claim 1, wherein the gripping member comprises a double helix, the double helix formed from two helices of opposite hands.

13. The device of claim 1, wherein the gripping member comprises a helical spring.

14. The device of claim 1, wherein the gripping member comprises a pattern of fenestrations.

15. The device of claim 1, wherein the gripping member comprises a braided structure.

16. The device of claim 1, wherein the gripping member comprises a woven structure.

17. The device of claim 1, wherein the gripping member comprises a tubular structure.

18. The device of clam 17, wherein said tubular structure is polymeric, and wherein said polymeric tubular structure is molecularly oriented to effect a diametrical contraction of the polymeric tubular structure upon the exertion of a longitudinal force in a first direction.

19. The device of claim 1, further comprising a deployment structure mounted for movement over the sheath member for positioning the gripping member onto the elongated structure.

20. The device of claim 1, further comprising a draw means connected to the gripping member and extending outwardly of the patient.

21. The device of claim 20, wherein the draw means is moveable in a first direction so as to collapse the gripping member about the encircled portion of the elongated structure and moveable in a second direction so as to expand the gripping member about the encircled portion of the elongated structure.

22. The device of claim 21, wherein the sheath is configured as a packaging system, wherein said packaging system is configured to maintain a geometry of the gripping member and the draw means prior to of the device.

23. The device of claim 1, further comprising a grasping means attached to the distal end of the gripping member for attaching the gripping member to the elongated structure.

24. The device of claim 1, wherein a longitudinal force applied to the gripping member in a first direction causes the gripping member to contract and wherein a longitudinal force applied to the gripping member in a direction opposite to the first direction causes the gripping member to expand.

25. The device of claim 1, wherein said gripping member is self-tightenable about the sheath member.

26. The device of claim 25, wherein the self-tightenable gripping member comprises a first configuration having a first diameter that is greater than the outside dimension of the elongated structure, and a second configuration having a second diameter that is about the same as the outside dimension of the elongated structure.

27. The device of claim 25, wherein the self-tightenable gripping member further comprises a proximal portion that extends outwardly beyond the proximal end of the elongated structure and outwardly of at least the vascular system of the patient.

28. A device for removing from a patient a previously implanted elongated structure; the elongated structure having an outside dimension, a proximal end, and a distal end located within the patient, the device comprising:
a) a sheath member having an inside dimension greater than the outside dimension of the elongated structure; and
b) a gripping member engaged with the sheath member, the gripping member having a proximal end, a distal end, a longitudinal passage extending between the proximal end and the distal end, the longitudinal passage dimensioned to encircle at least a portion of the elongated structure, and a proximal portion that extends outwardly beyond the proximal end of the elongated structure and outwardly of at least the vascular system of the patient,
wherein the gripping member comprises a first configuration having a first diameter that is greater than the outside dimension of the elongated structure, and a second configuration having a second diameter that is substantially the same as the outside dimension of the elongated structure.
